(19) United States
(12) Patent Application Publication
(10) Pub. No.: US 2014/0074104 A1
(43) Pub. Date: Mar. 13, 2014

(54) BONE CEMENT INJECTOR ASSEMBLY AND METHOD OF USE

(71) Applicant: VOT, LLC, O' Fallon, MO (US)

(72) Inventors: David BLAKEMORE, Cassopolis, MI (US); Thomas CARTER, Wentzville, MO (US); Eric LUCAS, Clemson, SC (US); Ross MACK, Valparaiso, IN (US); Shaun PITTS, Warsaw, IN (US); Ronald CLARK, Yuma, AZ (US)

(73) Assignee: VOT, LLC, O' Fallon, MO (US)

(21) Appl. No.: 14/082,980

(22) Filed: Nov. 18, 2013

Related U.S. Application Data
(63) Continuation of application No. 12/960,253, filed on Dec. 3, 2010.
(60) Provisional application No. 61/266,366, filed on Dec. 3, 2009.

(51) Int. Cl. A61B 17/88

(52) U.S. Cl. CPC A61B 17/8816; A61B 17/8841
USPC 606/94

(57) ABSTRACT

An injector assembly for delivering flowable material to a medical implant, including a tube having a first end and a second end, a connector member and a port connector, where the connector member is coupled to the first end of the tube and the port connector is coupled to the second end of the tube, the connector member being configured to attach to a material delivery mechanism and the port connector being configured to attach to the medical implant. When the material delivery mechanism is actuated, the flowable material passes through the tube to the medical implant. A method of using an injector assembly to deliver a flowable material to a medical implant and bone surface is also described. A kit containing various sized parts for an injector assembly is also described.

### CROSS-REFERENCE TO RELATED APPLICATIONS

This application is a continuation of U.S. application Ser. No. 12/960,253 filed Dec. 3, 2010, which claims priority to U.S. Provisional Application No. 61/266,366 filed Dec. 3, 2009, each of which are hereby incorporated herein by reference in their entireties.

### TECHNICAL FIELD

The present invention relates generally to surgical instrumentation, and more specifically, but not exclusively, to orthopaedic and neurosurgical instrumentation and techniques used for the injection of bone cement or bone filler material.

### BACKGROUND OF THE INVENTION

Typical orthopedic and neurological device procedures rely on easily manipulated viscous or doughy cement positioned upon the medical implant prior to placing the device against the prepared bone. It is desirable to inject less viscous or “thin” cement behind the implant or, alternatively between the implant and the host bone after the medical implant is positioned on the resected portion of the bone. This “thin” cement is difficult to handle so it must be contained to prevent excessive extrusion and to provide pressurization and interdigitation into the porous bone and medical implant geometries for complete fixation. The liquid or “thin” cement described does not hold shape and, therefore must be contained as well as routed through distinct structural geometries in order for it to be usable by the surgeon. Further, the adhesive properties of cement while in this liquid state are such that it cannot be handled because of the difficulties associated with it smearing and adhering to any surface the material comes in contact with. A need is present for the development of an instrument that will allow the user to manipulate and control the flow of the liquid bone cement or filler during the implantation of medical devices that use such material in this state.

### SUMMARY OF THE INVENTION

Advancement of the state of the surgical instrumentation that is used to control and inject bone cement and filler is desirable. The embodiment of the invention described herein satisfies the need for improvements to surgical instruments used to precisely deliver bone cement in a less viscous state to the implant/bone interface.

The present invention provides in one aspect, an injector assembly for delivering a flowable material to a medical implant, the assembly includes a tube having two ends, and a connector member that is attached to one of the ends. The injector assembly also has a port connector attached to the other end of the tube. The connector member is constructed to detachably attach to a material delivery mechanism or syringe like device and the port connector is constructed to mate with the medical implant. When the material delivery mechanism is actuated, the flowable material will pass through the tube to the medical implant via the port connector.

In another aspect of the invention, the connector member is detachably coupled to the first end of the tube. In another aspect of the invention, the port connector is detachably connected to the second end of the tube. In an alternative embodiment of the invention, the port connector also includes a means for securement to the medical implant. In one embodiment, the means for securement to the medical implant includes at least one tip. In another embodiment of the invention, the tip may be compressible, or tapered or both. In another embodiment of the invention, the tip includes a plurality of slits. In another aspect of the invention the port connector is straight to facilitate connecting to the medical implant. In another aspect of the invention the port connector is angled to facilitate connecting to the medical implant. In another embodiment of the invention the port connector is fixed or adjustable. In another aspect of the invention, the tube is fabricated from a flexible material such that the tube will absorb external forces and movements, maintain the connection between the delivery mechanism and the medical implant. In another aspect of the invention the tube includes at least one internal channel which is configured to facilitate the delivery of a flowable material, gas, air or suction. In another aspect of the invention, the tube is configured to facilitate the contraction or expansion of the internal channel. In another aspect of the invention the injector assembly includes a second tube with a first end and a second end, where the connector member is connected to the first end and the port connector is connected to the second end.

The present invention provides in yet another aspect, method for delivering flowable material to a medical implant. The method may include the steps of obtaining an injector assembly that has a tube with two ends, a connector member that attaches to one of the tube ends and a port connector that attaches to the other opposite end of the tube. The connector member is built to attach to a material delivery mechanism and the port connector is constructed to connect to the medical implant. The method may further include the step of attaching the port connector to the medical implant. The method may further include the steps of placing the medical implant onto a bone and then attaching the connector member to the material delivery mechanism. The method may also include the step of actuating the material delivery mechanism to inject the flowable material into the injector assembly.

Yet a further aspect of the present invention provides a flowable material injector kit. The kit may include a multitude of tubes. The tubes all having various sizes, diameters, flexibilities, and lengths. The multitude of tubes may also have a single internal channel or multiple internal channels. The kit may also include a plurality of various sized and angled port connectors. The port connectors may have fixed angled tips or variable angled tips. The bone cement injector kit may also have multiple connector members, each being of a different size and diameter, as well as having a different mating configuration. The kit further includes a plurality of various sized pressurized reservoir devices.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the end of the specification. The foregoing and other objects, features, and advantages of the invention are apparent from the following detailed description taken in conjunction with the accompanying drawings in which:

FIG. 1 is a perspective view of one embodiment of the injector assembly, in accordance with an aspect of the invention;

FIG. 2 is a perspective view of an alternative embodiment of the injector assembly of FIG. 1, in accordance with an aspect of the invention;

FIG. 3 is a perspective view of an alternative embodiment of the injector assembly of FIG. 1, in accordance with an aspect of the invention;

FIG. 4 is a perspective view of an alternative embodiment of the injector assembly of FIG. 1, in accordance with an aspect of the invention;

FIG. 5 is a perspective view of an alternative embodiment of the injector assembly of FIG. 1, in accordance with an aspect of the invention;

FIG. 6 is a perspective view of an alternative embodiment of the injector assembly of FIG. 1, in accordance with an aspect of the invention;

FIG. 7 is a perspective view of an alternative embodiment of the injector assembly of FIG. 1 where the port connector has been coupled to a medical implant, the medical implant has been placed onto a bone, and the connector member has been coupled to a material delivery mechanism, in accordance with an aspect of the invention; and

FIG. 8 is a flow chart of a method of using the injector assembly of FIG. 1, in accordance with an aspect of the invention.

### DETAILED DESCRIPTION FOR CARRYING OUT THE INVENTION

For the purposes of promoting an understanding of the principles of the injector assembly, reference will now be made to the embodiments, or examples, illustrated in the drawings and specific language will be used to describe these. It will nevertheless be understood that no limitation of the scope of the invention is thereby intended. Any alterations and further modifications in the described embodiments, and any further applications of the principles of the invention as described herein are contemplated as would normally occur to one skilled in the art to which injector assembly invention relates.

In this detailed description and the following claims, the words proximal, distal, anterior, posterior, medial, lateral, superior and inferior are defined by their standard usage for indicating a particular part of a surgical instrument or surgical opening according to the relative disposition of the surgical instrument, surgical opening or directional terms of reference. For example, “proximal” means the portion of the surgical instrument positioned nearest the torso while “distal” indicates the part of the surgical instrument farthest from the torso. As for directional terms, “anterior” is a direction towards the front side of the body, “posterior” means a direction towards the back of the body, “medial” means towards the midline of the body, “lateral” is a direction towards the sides or away from the midline of the body, “superior” means a direction above, and “inferior” means a direction below another object or structure.

As used herein, the terms “surgical instrument,” “injector,” “injector assembly,” and “assembly” may be used interchangeably as they essentially describe the same type of operative instrument. In addition, the terms “bone cement,” “bone filler,” “filler,” and “cement” may be used interchangeably as they essentially describe the same type of material that is used to occupy a space or gap.

Generally stated, disclosed herein is a surgical instrument or injector assembly for use as a liquid cement/filler delivery device for bone cement, which for example purposes may include polymethylmethacrylate, or other flowable materials, like antibiotics, bone growth compounds, and bone matrixes that allows the surgeon to precisely deliver the filler in a less viscous state to the implant/bone interface. The surgical instrument interfaces with a syringe or other reservoir type of device and provides a closed pathway for the cement/filler to travel to the medical implant.

Further, described herein is a surgical method for using the injector assembly and a material injector kit that is used to deliver flowable substances into the body.

Generally, the injector assembly provides an interface for the material filled syringe or other like injector device on one end and the medical implant ports on the other end. The material is passed through the injector assembly on its way to being delivered to the underside or inside of the medical implant. The injector assembly has an internal geometry that is large and smooth enough, so as not to impart excessive friction on the material as it is passed through the injector assembly. Since the injector assembly may be put in place prior to material delivery and can remain in place long after the proper fill is achieved, it also may be configured to avoid jarring or mal-positioning of the medical implant from external factors or forces placed on the assembly. A flexible tube may be used in the injector assembly to cushion or absorb any external forces or movements that might occur during the surgical procedure. This flexible tube may be modular to allow for length, girth and flexibility adjustment.

Functionally, the injector assembly provides the user with a continuous means of delivering the liquid filler or cement to the underside or backside surfaces, as well as possibly, to an inside compartment of the medical implant and also to the bone substrate/surface of the patient.

Typical orthopedic or neurological device procedures rely on easily manipulated viscous or doughy cement positioned upon, or alternatively inside the implant prior to placing the implant against the prepared bone surface. A less viscous or “thin” cement is used with the injector assembly and is injected behind the medical implant after the implant has been positioned on the resected surface of the bone. This “thin” cement or filler is difficult to handle so it must be restricted and contained to prevent excessive extrusion around the medical implant during and after injection and to provide pressurization and interdigitation into the porous bone and medical implant geometries to achieve complete fixation.

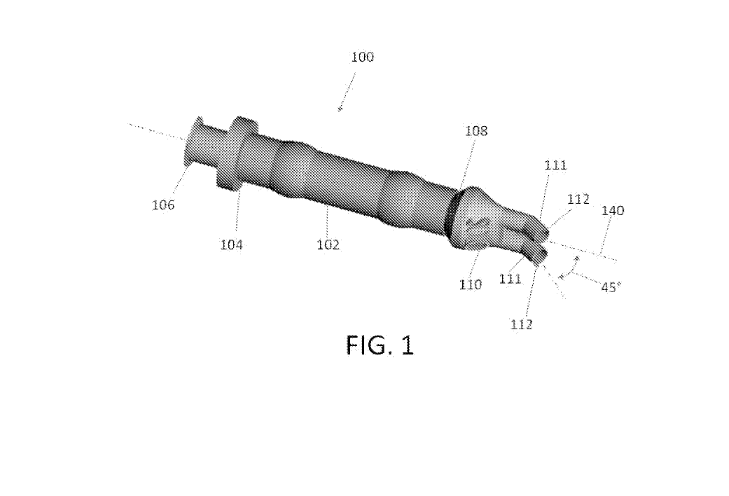

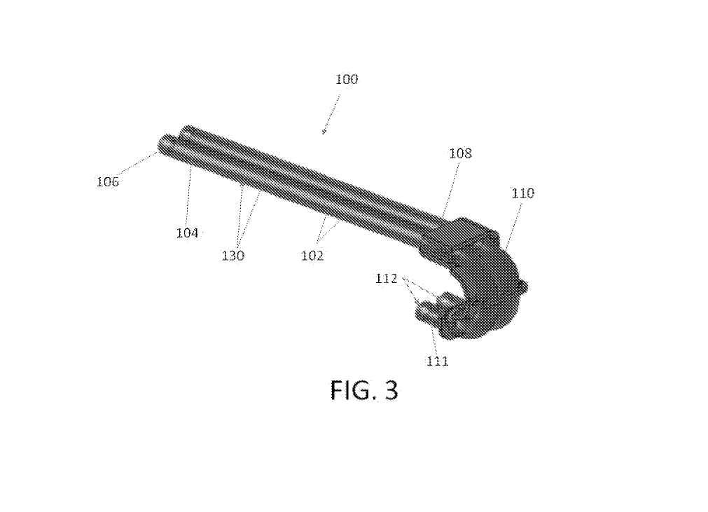

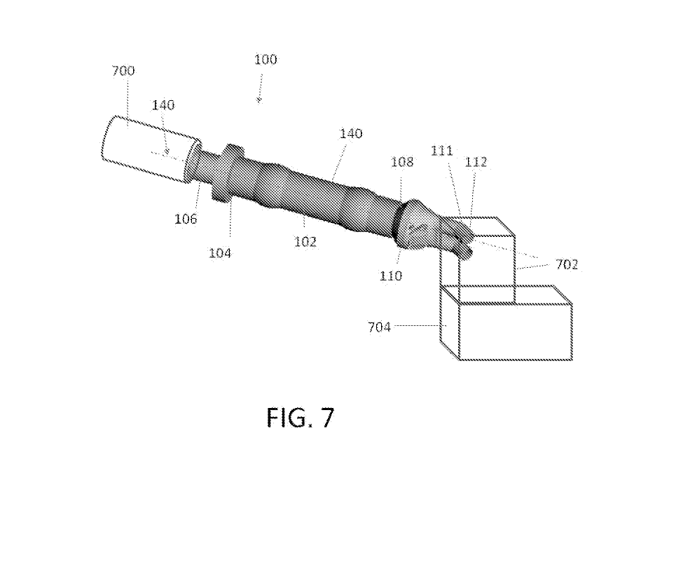

Referring now to FIG. 1, an injector assembly 100 in accordance with one aspect of the invention is shown. Injector assembly 100 includes a tube 102, which allows the passage of bone cement or other flowable material to flow through injector assembly 100. A connector member 106 is coupled to a first end 104 of tube 102 and is configured to couple to a pressurized reservoir device 700 (shown in FIG. 7), examples of which include but are not limited to syringes, pipettes, and other known pressurized reservoir devices. A port connector 110 is coupled to a second end 108 of tube 102. Port connector 110 may include a means of securement to a medical implant, an example of which is one or more tips 111. One or more tips 111 may be located at the distal end of port connector 110. Port connector 110, and more specifically one or more tips 111 are configured to interface with a medical implant, a portion of anatomy, specifically a bone surface, or both. The internal passage 130 (shown in FIG. 3) of tube 102, first end 104, connector member 106, second end 108, and port connector 110 are each configured with sufficiently large cross sectional areas to provide for a smooth passage of the flowable material.

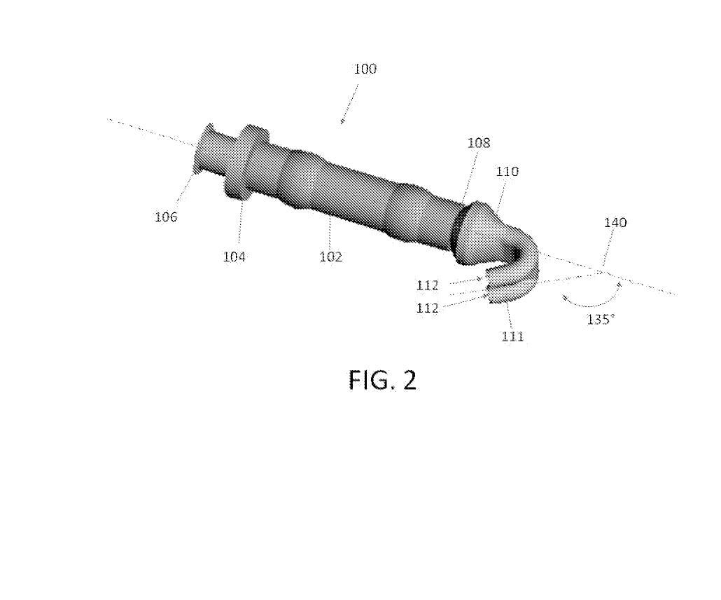

Now referring to FIGS. 1 and 2, port connector 110 is configured with one or more ports 112, to provide an enclosed passage from the injector assembly 100 to a portion of anatomy, implant, or both. Port connector 110 may be detachably coupled to second end 108. For example purposes, port connector 110 may be coupled with a hose barb, a threaded screw, a snap fit, a press fit, or other known means of detachably mounting. In another embodiment, port connector 110 may be permanently coupled to second end 108 as shown in FIGS. 3-6. In order to provide improved access to a surgical site, the one or more tips 111 may be angled or curved away from a central axis 140 of tube 102 to varying degrees and configurations. For example purposes, FIG. 1 depicts port connector 110 where the one or more tips 111 are angled at 45° away of offset from central axis 140. FIG. 2 depicts port connector 110 where the one or more tips are angled at 135° away from central axis 140. These angles should only be considered representative as angles between 45° and 135° may be used. Alternatively, an angle less than 45° or greater than 135° may be selected. The angle is selected by the surgeon to provide the best access to the surgical site. In another embodiment, port connector 110 and one or more tips 111 are flexible, and may be manipulated in order to provide the preferred angle of deflection away from central axis 140. For example, a force may be applied to the distal end of port connector 110, and one or more tips 111 shown in FIG. 1 in order to deform the port connector 110 and one or more tips 111 to the position shown in FIG. 2. Alternatively, port connector 110 and one or more tips 111 may be manipulated after the injection assembly 100 is brought into proximity with the medical implant or portion of anatomy or both.

Still referring to FIGS. 1 and 2, port connector 110 has a slight taper on the distal end. The outer diameter of the port connector 110 decreases as the one or more ports 112 are approached. The taper allows the user to mate port connector 110 with the corresponding geometry and opening located on a medical implant. The degree of the taper is very slight and may be approximately 1 to 10 degrees. Alternatively, or in combination with the taper, the one or more tips 111 may be comprised of a material capable of compression or expansion to allow for insertion into the medical implant.

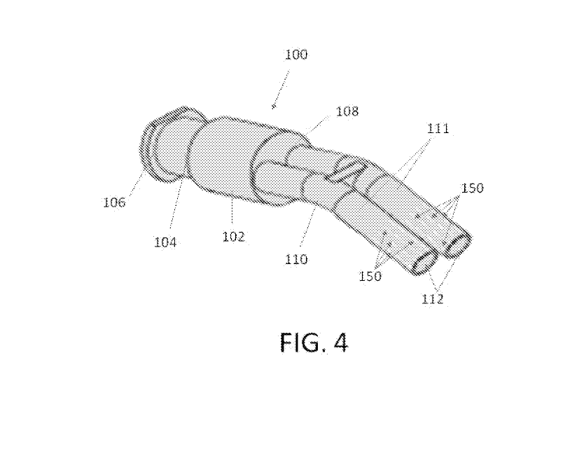

Referring now to FIG. 4, as an alternative or in combination with the taper, the port connector 110 may be provided with one or more slits 150, cuts or other apertures. In one embodiment, the one or more slits 150, cuts or apertures are provided in, on or near the distal end of port connector 110, and may be located on the one or more tips 111. The one or more slits 150, cuts or apertures allow for the compression or expansion of port connector 110, specifically the distal end thereof, as the port connector 110 matingly engages a medical implant or portion of anatomy or both.

Referring now to FIG. 3, more than one tube 102 may be provided, thereby creating more than one fluid path in communication with the connector port 110. In the embodiment where more than one fluid path or tube 102 is provided, one or more tubes 102 allows for the introduction of fluids and flowable materials, and one or more tubes 102 provides for a connection to a vacuum or suction source. In still another embodiment, one or more internal channels may be formed within tube 102 to allow for the introduction of multiple flowable materials and/or a connection to a vacuum source.

In the embodiments described above and below, the connector port 110 specifically, and the injector assembly 100 generally is comprised of a biocompatible material such as, for example purposes, metal or plastic. The biocompatible material being sterilizable. Any known sterilization technique may be used; contemplated examples include, but are not limited to conventional gamma ray, E-beam, ETO chemical and steam sterilization.

Injector assembly 100 may be put in place prior to material delivery and can remain in place after the proper fill is achieved. Therefore, injector assembly 100 may be capable of tolerating jarring or mal-positioning of the medical implant from external factors/forces. Tube 102, which may be fabricated from a flexible material, is also configured to cushion or absorb any external forces or movements that might occur during the surgical procedure while port connector 110 is coupled to the medical implant. Tube 102 as shown is generally flexible, but it should be understood that rigid or semi-rigid transport tubing may be used depending upon the given clinical situation.

It is contemplated that injector assembly 100 may be modular in design, as shown in FIGS. 1 and 2. Various connector members 106 may be coupled to first end 104 of tube 102. In addition, tube 102 may be selected from a variety of tubes 102, depending on the desired length, diameter and flexibility. Various sized and angled port connectors 110 with varying port 112 sizes, dimensions and cross sectional shapes may be removably coupled to second end 108.

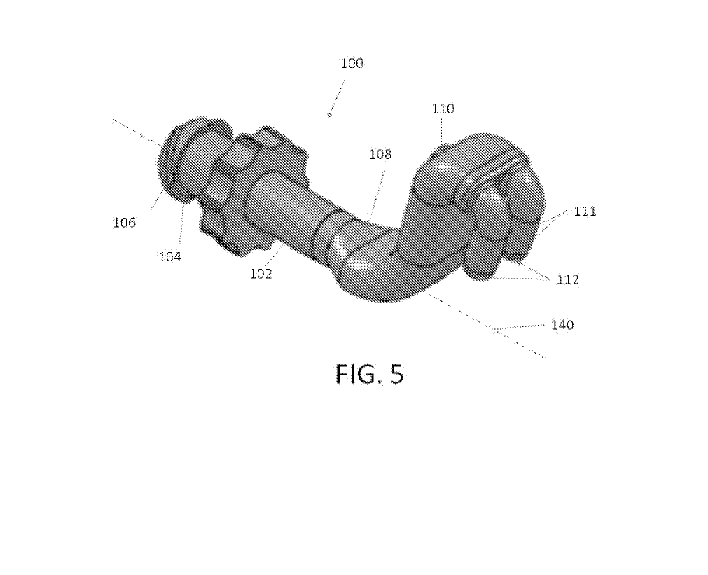

Referring now to FIGS. 3-6, injector assembly 100 may also be fabricated as a single molded piece. Thus, in these embodiments tube 102 is permanently coupled to connector member 106 at first end 104 and port connector 110 at second end 108. Referring now specifically to FIG. 4, a permanently coupled embodiment injector assembly 100 is shown in a generally rigid configuration. Referring now to FIG. 5, port connector 110 is shown as raised. Generally speaking, port connector 110 may be configured such that the path of the flowable material is non-coaxial with central axis 140 of tube 102.

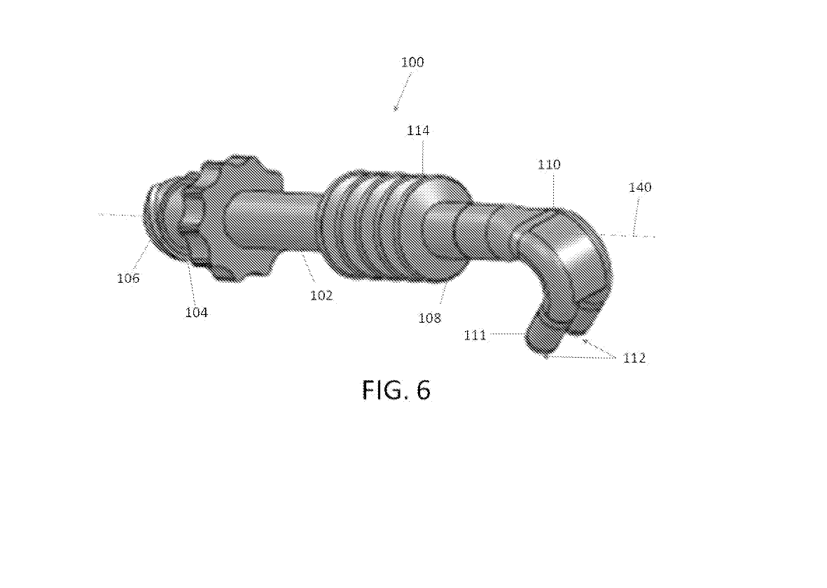

Now referring to FIG. 6, injector assembly 100 may include a bellows 114, or similar construct, which provides flexibility to allow manipulation before and/or after the flowable material is applied to the medical implant. As shown in FIG. 6, tube 102 may include bellows 114. Alternatively, port connector 110 may include bellows 114 to provide for increased flexibility and access at the distal aspect of injector assembly 100. Bellows 114 is one example of how tube 102 may be configured to facilitate implantation, contraction and expansion of the internal channel. Alternatives to the bellows may include sliding sleeves, a compressible material, and a twistable material.

Injector assembly 100, and more specifically, tube 102 may be configured to allow the user to deliver bone cement, other types of fillers in a less viscous state and flowable material to the implant/bone interface, as well as various other fluid materials and gasses, such as, for example purposes, antibiotics, saline, nitrogen, and carbon dioxide. In addition, injector assembly 100 is also capable of providing a passage for applying a vacuum or suction directly to the implant and bone interface. The additional fluids, gasses, vacuum and suction operations allow the user to irrigate, clean and prep the implant, bone, and bone/implant interface prior to cementing and/or filling the implant, bone and/or bone/implant interface.

Generally speaking, the method of use of injector assembly 100 includes the steps of the user securing the one or more tips 111 of the connector port 110 to the medical implant such that there is no leaking of appreciable amounts of flowable material. The user then places the medical implant with the injector assembly 100 on to the resected bone. Alternatively, the user may initially seat the medical implant onto the resected bone and subsequently secure the injector assembly 100 to the implant. The user then attaches a syringe, pipette or other pressurized reservoir device filled with flowable material to the connector member. The user may also verify that the flowable material is prepared. The syringe or other reservoir device is then actuated, thereby forcing the flowable material from the reservoir, and the volume of flowable material is monitored as the implant cavities and bone porosity are filled. Following the introduction and insertion of the appropriate amount of flowable material, but prior to the curing or hardening of the flowable material, the user will then remove the injector assembly and attached syringe from the medical implant.

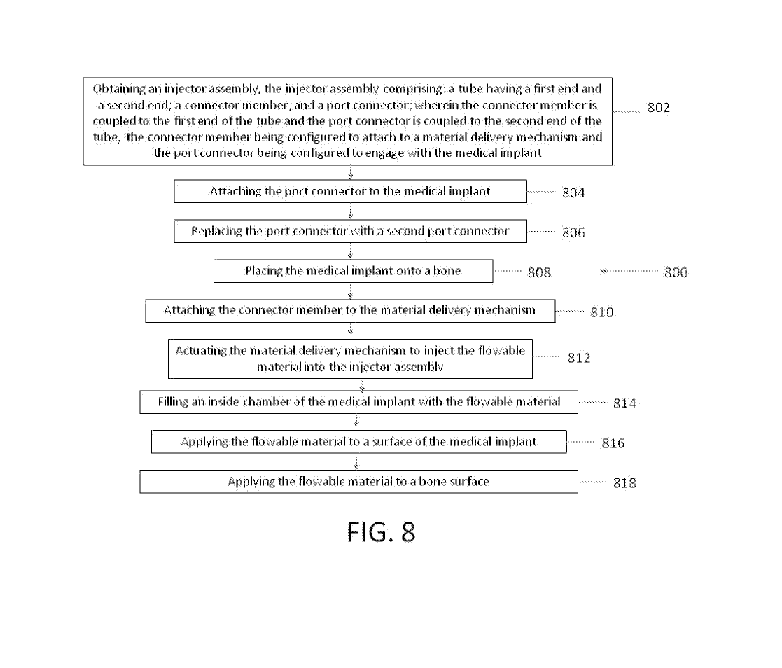

Referring now to FIG. 8, the method 800 of delivering flowable material to a medical implant is shown. Specifically, the method 800 includes step 802 of obtaining an injector assembling, the injector assembly including, a tube having a first end and a second end, a connector member, and a port connector, where the connector member is coupled to the first end of the tube and the port connector is coupled to the second end of the tube, the connector member being configured to attach to a material delivery mechanism and the port connector being configured to engage the medical implant. The method 800 also includes step 804 of attaching the port connector to a medical implant, step 808 of placing the medical implant onto a bone, step 810 of attaching the connector member to the material delivery mechanism, and step 812 of actuating the material delivery mechanism to inject the flowable material.

In one alternative, the method 800 may also include the step 806 of replacing the port connector with a second port connector. Step 806 allows the surgeon or user to select a different port connector, which provides better access to the medical implant. In another alternative method 800 includes the step 814 of filling an inside chamber of the medical implant with the flowable material. In another alternative, method 800 includes the step 816 of applying the flowable material to a surface of the medical implant. In still another alternative, method 800 includes the additional step 818 of applying the flowable material to a bone surface.

After completing step 802, injection assembly 100 is obtained such as the one shown in FIG. 1. For illustrative purposes, FIG. 7 shows a injection assembly 100 where step 804 has been completed, and the port connector 110 has been attached to a medical implant 702. The completion of step 810 is also shown where the connector member 106 is attached to a flowable material delivery mechanism 700. The medical implant 702 has been placed onto a bone 704.

It is also contemplated that the injector assembly may be included in a material injector kit. The kit may have various sizes, diameters, flexibilities, and lengths of the single channel and multiple channel tubes. Also included in the kit may be various sizes and angulation of the connecting tips. The orifices of the connecting tips may have fixed angles or variable angles. In addition, the kit may have different connectors for coupling to the syringe or other material reservoirs. Further still, the kit may include various sized material delivery syringes or other pressurized reservoir devices. The injector assembly may be available within the kit as a one piece device or modular, in that the user builds their own from the above noted individual components, all embodiments of the injector assembly in the kit will typically include the above described construct elements that for brevity sake, will not be discussed again here and include the same structural and functionality characteristics as described previously herein.

Although the various embodiments have been depicted and described in detail herein, it will be apparent to those skilled in the relevant art that additional modifications, and substitutions can be made without departing from its essence and therefore these are to be considered to be within the scope of the following claims.

What is claimed is:

1. An injector assembly for delivering flowable material to a medical implant, the assembly comprises:
a tube configured to facilitate contraction and extension, wherein the tube comprises a first end and a second end;
a connector member; and
a port connector;
wherein the connector member is coupled to the first end of the tube and the port connector is coupled to the second end of the tube, the connector member being configured to attach to a material delivery mechanism and the port connector being configured to couple to the medical implant, wherein when the material delivery mechanism is actuated the flowable material passes through the internal channel of the tube to the medical implant.

2. The injector assembly of claim 1, wherein the connector member is detachably coupled to the first end of the tube.

3. The injector assembly of claim 1, wherein the port connector is detachably connected to the second end of the tube.

4. The injector assembly of claim 1, wherein the port connector further comprises a means for securement to the medical implant.

5. The injector assembly of claim 4, wherein the means for securement to the medical implant comprises at least one tip.

6. The injector assembly of claim 5, wherein the at least tip is at least one of compressible and tapered.

7. The injector assembly of claim 5, wherein the at least one tip further comprises a plurality of slits.

8. The injector assembly of claim 1, wherein the port connector is straight to facilitate connecting to the medical implant.

9. The injector assembly of claim 1, wherein the port connector is angled to facilitate connecting to the medical implant.

10. The injector assembly of claim 9, wherein the angle of the port connector is at least one of fixed and adjustable.

11. The injector assembly of claim 1, wherein in the tube is fabricated from a flexible material such that the tube will absorb forces and maintain the connection between the material delivery mechanism and the medical implant.

12. The injection assembly of claim 1, wherein the tube comprises at least one internal channel, wherein the at least one internal channel is configured to facilitate the delivery of at least one of a flowable material, gas, and suction.

13. The injector assembly of claim 5, wherein the tube further comprises a central axis extending longitudinally through the center of the tube from the first end to the second end and the port connector further comprises a proximal end and a distal end, wherein the means for securement is positioned at the distal end of the port connector and the at least one tip of the means for securement is curved away from the central axis of the tube and includes at least one opening.

14. The injection assembly of claim 1, further comprises at least a second tube, the at least a second tube having a first end and a second end, wherein the connector member is connected to the first end and the port connector is connected to the second end.

15. A method for delivering flowable material to a medical implant, the method comprising:
obtaining an injector assembly, the injector assembly comprising;
a tube configured to facilitate movement, wherein the tube comprises a first end and a second end;
a connector member; and
a port connector;
wherein the connector member is coupled to the first end of the tube and the port connector is coupled to the second end of the tube, the connector member being configured to attach to a material delivery mechanism and the port connector being configured to engage with the medical implant;
attaching the port connector to the medical implant;
placing the medical implant onto a bone;
attaching the connector member to the material delivery mechanism; and
actuating the material delivery mechanism to inject the flowable material into the injector assembly.

16. The method of claim 15, further comprising:
replacing the port connector with a second port connector.

17. The method of claim 15, further comprising:
filling an inside compartment of the medical implant with the flowable material.

18. The method of claim 15, further comprising:
applying the flowable material to a surface of the medical implant.

19. The method of claim 15, further comprising:
applying the flowable material to a bony surface.

20. A material injector kit, the kit comprising;
a plurality of tubes having various sizes, diameters, flexibilities, and lengths of single internal channel tubes and multiple internal channel tubes;
a plurality of various sized and angled port connectors;
a plurality of various sized connector members; and
a plurality of various sized pressurized reservoir devices.
